(19) United States
(12) Patent Application Publication
(10) Pub. No.: US 2010/0036391 A1
(43) Pub. Date: Feb. 11, 2010

(54) METHOD FOR RAPID AND ACCURATE ENTRY THROUGH SOFT TISSUE AND BONE

(76) Inventors: EDWARD R. ZALESKI, Santa Ana, CA (US); Edward Wong, Newport Beach, CA (US); Michael T. Vu, Newport Coast, CA (US); Markus Wong, Newport Beach, CA (US); Robert White, Shaker Heights, OH (US)

Correspondence Address: WALTER A. HACKLER, 2372 S.E. BRISTOL, SUITE B, NEWPORT BEACH, CA, 92660-0755, US

(21) Appl. No.: 12/548,357

(22) Filed: Aug. 26, 2009

Related U.S. Application Data
(62) Division of application No. 11/159,478, filed on Jun. 23, 2005, now abandoned.
(60) Provisional application No. 60/582,618, filed on Jun. 24, 2004.

(51) Int. Cl. A61B 17/56

(52) U.S. Cl. 606/108; 128/898

(57) ABSTRACT

A method for entering soft tissue and bone of a body, the method includes sterilizing a scalp, injecting a spike, with a grommet thereabout, through a calvaria, and removing the spike and leaving the grommet inserted in the calvaria, the grommet providing intracranial access.

The present application is a division of U.S. Ser. No. 11/159,478 filed Jun. 23, 2005, now abandoned which claims priority from U.S. Provisional Patent Application Ser. No. 60/582,618 filed Jun. 24, 2004. This application is to be incorporated herewith in toto by the specific referenced thereto.

The present invention generally relates to medical devices useful in rapidly entering through soft tissue and bone of the body. Specific application to the central nervous system (CNS) includes the treatment of intracranial diseases, including subdural hematoma, epidural hematoma, increased intracranial pressure, and other diseases requiring rapid entry into the lateral ventricle or other intracranial space or subarachnoid space. The invention also provides devices for insertion into the lateral ventricle(s) of the brain and the space (including the subarachnoid space) surrounding the spinal cord for use in hypothermia or hyperthermia applications, the exchange of cerebral spinal fluid (CSF), the application of treatment modalities, and the insertion or a ventriculostomy unit. The use of stereotaxic guidance in a controlled environment or the use of a helmet for stereotaxic control when needing emergent treatment will aid in accurate placement of the unit. The medical device has a powered unit capable of inserting a sharp device through the soft tissue and bone of the head rapidly, producing a self-sealing mechanical grommet through the skull, and allowing for rapid evacuation of fluid from the epidural space, the subdural space, or the subarachnoid space. If a ventriculostomy into the lateral ventricle is desired, it can be passed through the grommet. A navigational system including ultrasound guidance through the tip of the inserter can give visual and auditory clues in order to enter the lateral ventricle as the inserter is placed through the dura, the arachnoid, and the brain tissue. A fiber optic camera may be configured to aid in visualization of the surrounding tissues, spaces, and lateral ventricle. The inserter has a plastic sheath around its shaft, and once the lateral ventricle or subarachnoid space is entered, the inserter is withdrawn out of the head, leaving the plastic sheath as a conduit for insertion of a probe into the lateral ventricle or subarachnoid space. Such a probe may have in its tip a thermister for temperature measurement, irrigation/aspiration ports for fluid exchange and application of therapeutic modalities, and a pressure manometer for pressure measurement.

Trauma to the brain and/or spinal cord may result in direct injury to the central nervous system (CNS) tissue as well as to swelling or edema of this tissue against the walls of the skull or spinal canal. In the case of hemorrhage, there may be compression of the brain or spinal cord from within or around the tissue. After a period of time (minutes to hours to days), death of the tissue may occur causing irreversible damage.

Pathology to the brain may occur due to blunt injuries, such as a blow to the head, resulting in hemorrhage within or around the brain and associated swelling of brain tissue. Stroke, tumor, or other intracranial disease may also cause hemorrhage or swelling of brain tissue. Diagnosis of these diseases is made after careful neurological examination that is confirmed by imaging procedures including M.R.I., C.T., and ultrasound studies of the brain. Unfortunately, it is often difficult to control injury to the brain using conventional neurosurgical means including medical and surgical intervention.

Spinal injury may occur in blunt trauma due to a direct blow or to coup-countercoup injury. There may be direct pressure placed on the spinal cord as a result of a fractured or dislocated vertebral body (—ies) or disc, resulting in sensory and motor deficits below the level of the lesion. The mechanism of spinal injury is often related to swelling of the tissues of the spinal cord. Immediate treatment must be administered to prevent or diminish the effects of spinal cord compression and tissue edema.

Spinal cord ischemia may also occur during or following surgery on the aorta, including abdominal aneurysm repair with a prosthetic graft. Motor, sensory, and autonomic functions may be severely compromised or lost if the spinal cord is made ischemic.

Current treatment for swelling of the brain or spinal cord is not always satisfactory. In the severely injured brain or spinal cord, medical therapy to control swelling is usually applied systemically resulting in high levels of medication in the rest of the body with very low concentrations reaching the brain or spinal cord. Surgical intervention to decompress the brain or spinal cord requires major intervention through opening the skull or spinal column to expose the area and prevent compression against the fixed volume of the bony walls. Ventriculostomy placing a tube into the lateral ventricle of the brain) is usually not performed acutely, and by the time it is used in the sub-acute phase, there may already be permanent damage to the brain.

In the case of cardiac arrest, exsanguination, stroke, etc., lack of circulation to the brain will result in brain death within five minutes. A new approach to preventing brain death has been developed and presented in this patent in order to increase survival after medical resuscitation efforts.

Hypothermia has proved encouraging in the recent literature for the purpose of decreasing oxygen consumption and for decreasing swelling of CNS tissue. Unfortunately, cooling of the entire body to cool the brain or spinal cord does have inherent dangers. The heart responds to hypothermia with arrhythmias, and the blood clotting mechanisms may be severely impaired resulting in hemorrhage. Moreover, cooling the body only results in a few degrees of cooling of the CNS. This may be due to protective mechanisms in the hypothalamus of the brain, or due to difficulty in heat/cold exchange between the blood and the brain.

With the new technologies now available, an alternate approach is described to control the temperature of the CNS as well as administer medications to the CNS directly in a continuous fashion.

The purpose of this patent is to present a new approach to rapidly enter the skull within moments of arrival of the emergency physician. This will be useful in treating subdural hematoma, epidural hematoma, intraventricular hemorrhage, brain and/or spinal cord trauma, and medical conditions that may threaten the survival of the patient due to brain death.

### SUMMARY OF THE INVENTION

The invention provides devices and methods useful in rapidly entering through the soft tissue and bone of the body. Specifically, it can provide access for instruments used in the treatment of intracranial diseases, including subdural hematoma, epidural hematoma, increased intracranial pressure, and other diseases requiring rapid entry into the lateral ventricle or other intracranial space or subarachnoid space. It may also be used in conjunction with cooling the brain and/or spinal cord prior to and during surgical intervention. In addition, the brain and/or spinal cord may have other thermoregulation including warming/heating the tissue before, during, and after therapeutic intervention. Moreover, the principles of this invention may be applied to other organs in the body for thermoregulation, hypothermia, and application of circulating fluids with/without pharmaceutical agents.

The embodiment of the device is used to rapidly enter the skull and soft tissues of the head in order to treat intracranial diseases including subdural hematoma, epidural hematoma, increased intracranial pressure, and other diseases requiring rapid entry into the lateral ventricle or other intracranial space or subarachnoid space. By entering the subarachnoid space, it is possible to administer fluid, medications, and cooling treatment to the brain and/or spinal cord without some of the side effects of systemic therapy. The device consists of a unit that will henceforth be termed the “inserter.” The inserter is designed to accurately produce an opening into the patient's soft tissue and skull bone within seconds after the scalp is shaved and made aseptic in a sterile field. This is accomplished with the use of a specifically-designed, self-contained insertion unit that utilizes a high compression spring, or other gas or hydraulic propulsion, or other method to insert a “spike” with “portal grommet” attached. The insertion of the spike and portal grommet should be easily administered by a qualified physician or trained medical personnel. The inserter resembles a handheld gun, but it may be configured into a fixation unit such as a stereotaxic system or helmet system. The inserter can accommodate a number of different sized inserter spikes and grommets in different combinations of length and diameters.

The portal grommet also supports geometry to interact with the inserter assembly for precise depth control and can also facilitate reed-type seals or other self-sealing device as well as other flexible instrumentation. A sharpened removable spike having a portal grommet attached is driven into position to a preset depth into the patient's soft tissue and skull bone.

Loading and cocking the Inserter Assembly is achieved by first installing the desired Inserter spike and Portal grommet into the inserter's slotted piston (FIG. 4).

The Inserter gun's piston is then drawn back to the cocking position making it ready for use (FIG. 5).

Setting the depth of the driving piston is achieved by adjusting the stage height to a predetermined setting. This is achieved by unlocking the inserter stage and moving it to the desired spike depth, then re-securing the stage to the inserter barrel. Other methods of setting the stage can be applied. However, the preferred method allows the spike depth to be set at the time of use.

The insertion gun is positioned onto the targeted area (FIG. 1). With sufficient force applied to the inserter to prevent recoil, the unit is then fired by pulling the trigger of the inserter (FIG. 2).

The insertion gun is then disengaged from the implanted spike and grommet. The Insertion Spike is then removed from the Portal Grommet thereby leaving a well-defined entry for an assortment of surgical instruments for insertion into the cranial cavity or other targeted areas of the body.

In order to ensure proper placement of the unit into the head, a stereotaxic unit can be configured when there is time to insert the unit under controlled circumstances such as in the operating room. When there is emergent need to insert the device rapidly, the stereotaxic unit may be configured into a fixation helmet with a stereotaxic device. With three points (e.g. two auditory canals and the maxilla) as a reference, the helmet will give the appropriate trajectory as well as the support for the device necessary for proper control. FIGS. 12A-C-14A-B

Prior to the insertion of the conduit, a stylet is inserted through the proximal end up to the tip of the distal end of the conduit to increase it's tensile strength for ease of insertion into the brain/lateral ventricle/subarachnoid space, much like the modern day insertion of a ventriculostomy tube. [FIG.8-9]

A navigational system incorporated into the unit may have an ultrasound device at its end in order to guide the instrument into the subarachnoid space and into the lateral ventricle. The stylet will be rigid enough to allow entrance through the dura mater, the brain substance, and the lateral ventricle.

Another configuration of a navigational system consists of a fiber-optic camera with a diameter of 0.1-4.0 mm, a length between 1-20 cm, and with a lens at the distal end. When this fiber-optic camera is inserted into the conduit, the lens at the distal end of the conduit allows the conduit to penetrate through the hole in the skull as well as the brain parenchyma to any region of the brain including the subarachnoid space and/or the lateral ventricle. After the conduit is inserted into the lateral ventricle and/or subarachnoid space, the fiber optic camera may be manipulated within the brain parenchyma or subarachnoid space or lateral ventricle to visualize the adjacent area for proper placement of the flexible conduit. When the flexible conduit is verified to be properly aligned within the brain, the inserter unit with a fiber optic camera is withdrawn from the proximal end and the conduit is secured to the skull and the surrounding grommet using modern day surgical techniques. Backflow of CSF should occur after the removal of the unit which ensures that the conduit is inserted into the lateral ventricle and/or subarachnoid space and has a direct connection between the ventricular system of the brain and the outside milieu. A one-way valve in place at the proximal end of the flexible conduit is also provided to prevent the continual drainage of CSF from the brain. In cases of brain herniation/movement, the conduit bends in the direction of herniation/movement, thus eliminating/minimizing CNS injury. Another configuration may consist of a combination of both ultrasound localization and fiber optic camera localization attached to the unit to enhance localization and placement of the conduit.

The conduit is placed into the lateral ventricle and the subdural/subarachnoid space, through separate units; or through a single unit with openings into both the lateral ventricle and the subdural/subarachnoid space. Once communication has been made to these locations, then evacuation of blood or other bodily fluids may be completed with an irrigation/aspiration unit. Administration of medications may be instilled (e.g., antibiotics, anti-inflammatories, steroids, etc.). Temperature control may be accomplished through the conduits(s) by irrigation/aspiration of artificial cerebrospinal fluid (CSF) or by re-circulating CSF.

With the hollow conduit securely in place, the second part of this device will be used to induce hypothermia/hyperthermia selectively within the intraventricular and/or subarachnoid space of the CNS. An example is a medical device that will be inserted through the proximal end of the flexible conduit and will comprise the cooling/heating, circulating, temperature and pressure monitoring components.

The invention relates to a medical device useful in rapidly inserting a unit into the skull or other subarachnoid/subdural space to rapidly place a venticulostomy-type unit into the head, thereby reducing and preventing brain injury or spinal cord injury in patients with head or neck trauma, strokes, tumors, and other intracranial diseases. More specifically, the invention provides devices for insertion into the lateral ventricle(s) of the brain and the space (including the subarachnoid space) surrounding the spinal cord for use in hypothermia or hyperthermia applications, the exchange of cerebral spinal fluid (CSF), the application of treatment modalities, and the insertion or a ventriculostomy unit. The medical device may have within its length a fiberoptic endoscope for visualization and localization, and/or an ultrasound unit to aid in safely localizing the subarachnoid/subdural and/or the lateral ventricle. An external system housed in a briefcase will house the inserter and the monitor to visualize the ultrasound and/or fiberoptic camera. Ultrasonic localization of the lateral ventricle coupled with the fiber-optic endoscope ensures rapid and accurate insertion of the instrument into the CSF of the lateral ventricle. The design of the instrument allows rapid transfer of learning skills for any neurological surgeon or emergency physician to perform the ventriculostomy with a minimum of training and with unparalleled accuracy within minutes.

### BRIEF DESCRIPTION OF DRAWINGS

The advantages and features of the present invention will be better understood by the following description when considered in conjunction of the accompanying drawings, in which:

FIG. 1: represents a frontal cross section of the human head showing the outer scalp and skull bone tissue. Additionally the brain is also represented with the lateral ventricle near the center of the human head. The inserter is shown in position ready for use to insert the portal spike assembly;

FIG. 2: represents the same frontal cross section of the human head as in FIG. 1. The inserter is also in the same position, however the portal grommet is shown in its seated state within the tissue and skull bone;

FIG. 3: Depicts the portal grommet assembly as well as the spike (trochar) and grommet in the disassembled state;

FIG. 4: Shows a cross section view of the inserter in its loaded position;

FIG. 5: Shows a cross section view of the inserter and trigger mechanism in its loaded position ready for use;

FIG. 6a: Provides a detailed cross section view of a grommet design with a flexible sealing cap;

FIG. 6b: Shows a detailed cross section view of the grommet with the flexible sealing cap installed;

FIG. 7a: Depicts a cross section view of the same portal grommet, a detachable positioning adapter and a movable catheter sleeve used for limited trajectory positioning about the center of the grommet length; FIG. 7a: also showing the grommet and detachable positioning adapter with the catheter sleeve in its assembled position and the means by which it is held in place;

FIG. 7b: Depicts the assembly of the detachable positioning adapter and the catheter sleeve at a proposed angle off the centerline axes when installed onto the grommet;

FIG. 8: Depicts a cross section view of the same portal grommet, a detachable positioning adapter and a movable ultra sound probe when engaged into and through the portal grommet inner diameter;

FIG. 9a: Shows an ultrasound probe having a curved tip and its relationship to the portal grommet;

FIG. 9b: Shows an ultrasound device 161 attached to the ultrasound probe;

FIG. 9c: Shows a fiberoptic camera device 162 attached to the probe;

FIG. 10: Shows the grommet with an extension seal incorporating a vacuum chamber;

FIG. 11: Depicts a grommet with a reed-type seal;

FIG. 12a-12c: Shows a template to target the intended grommet location by use of the upper teeth (maxilla) as a point of reference;

FIG. 13: Shows a method of targeting with the unit in position;

FIG. 14a: Shows a frontal view of a stereotactic device attached to the helmet;

FIG. 14b: Shows a lateral view of a stereotactic device attached to the helmet; and

FIG. 15: illustrates the use of a vent elsewhere in the CNS, such as the subarachnoid space of the lumbar region, to allow for proper flow of fluid in the brain.

### DETAILED DESCRIPTION OF THE INVENTION AND PREFERED EMBODIMENTS

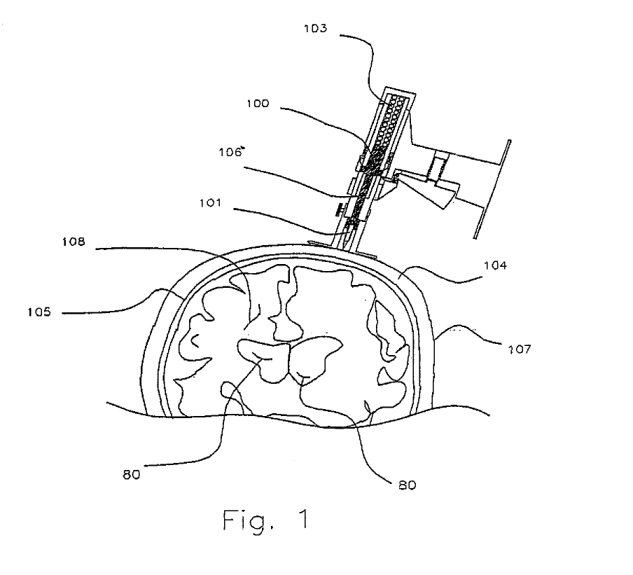

The invention will now be described with reference to a preferred embodiment thereof in FIG. 1.

The inserter comprising of a piston 100 is in contact with a compression spring 103 that supplies the necessary force required to propel a grommet/spike (trochar) assembly 101 forward through scalp tissue 104 and continuing through a skull 105. A piston stop 106 provides the necessary mechanism to stop the piston from advancing too far.

Further described in FIG. 1 are brain tissue 108 having lateral ventricles 80.

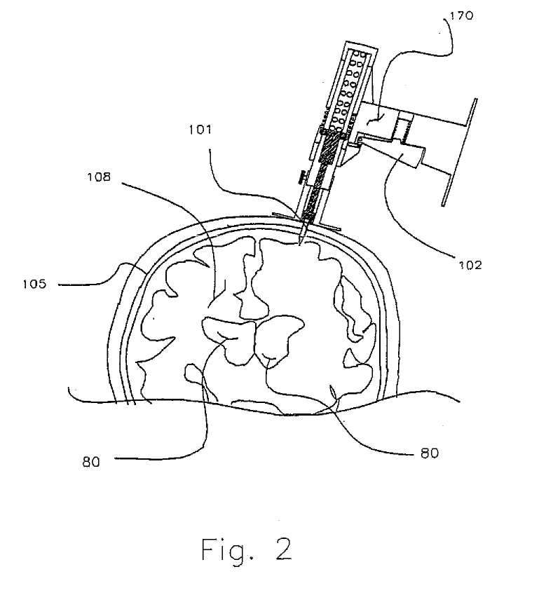

Advancement of the grommet/spike (trochar) assembly 101 is achieved by applying manual force on a trigger 102 as shown in FIG. 2.

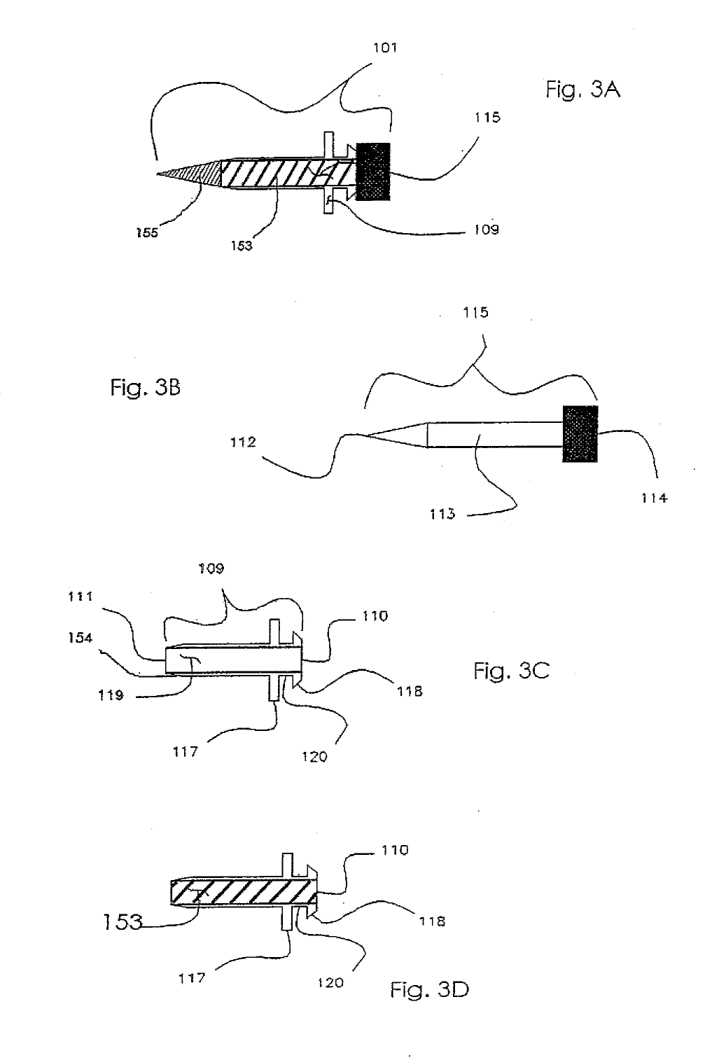

As can be seen in FIG. 3a, the grommet/spike (trochar) assembly 101 is comprised of a grommet 109 having a proximal end FIG. 3c 110 and a distal end 111 with the distal end having a sharp tapered edge 154 to improve passage through tissue as well as to facilitate case of insertion through the skull. The grommet unit has a spiral profile 153 as seen in FIG. 3d to enhance penetration while minimizing bone splintering or fracturing. The spiral profile will also facilitate removal of the grommet unit by allowing it to be unscrewed from the surrounding bone.

Also as shown in FIG. 3b is a spike (trochar) 115 comprising a sharp distal tip 112, a spike shaft 113, and a spike head 114. The spike distal tip 112 may support different configurations to optimize its ability to penetrate different tissues. Distal tip configuration may include, but not be limited to round circular configurations, multiple flats configurations, such as two-face flats, three-face flats or more faces, The taper configuration may start at the spike distal tip 112 and progress to the spike shaft diameter 113. The configuration can be made to include single and or multiple configurations to optimize the spikes ability to penetrate an assortment of biological tissues including animal and human tissues. The spike (trochar) 115 may have a serrated tip 155 with one or more facets to minimize bone splintering and tissue shearing.

The grommet 109 shown in FIG. 3c consists of a distal end 111 and can be tapered to facilitate ease of insertion through biological tissue. A grommet shaft 116 is of a suitable length and diameter to allow an entry into biological cavities. A hollow shaft of the grommet 119 may be of a different size in diameter including 0.01 mm to 50 mm or more to facilitate desired instrumentation to pass through the inner grommet to the intended inner cavity. The grommet supports a flange 117 that is used to prevent further advancement of the grommet into the intended biological tissue. An attachment flange 118 is suitably designed to provide a standardized means to secure various instruments and has a suitable distance from flange 117. This flange spacing 120 is used as an attachment point for grommet extraction devices.

As in FIG. 3b spike 115 is positioned into grommet 109 to complete the grommet/spike (trochar) assembly 101 which in turn can be inserted into the biological tissue.

### Insertion

The preferred method to insert the grommet/spike assembly 101 is to penetrate the biological tissue at a speed and force sufficient to exceed the resistance mass of the tissue body.

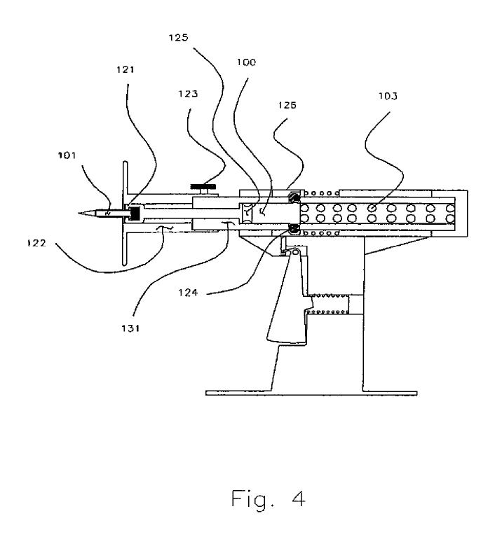

A preferred method to propel the grommet/spike assembly 101 into the tissue is illustrated in FIG. 4. Loading of the spike assembly can be achieved by insertion of the spike assembly 101 onto the spike-assembly retention cap that is secured to the piston 100. The spike retention cap 121 is suitably designed to contain the spike assembly 101 during rapid acceleration and deceleration which thereby provides controlled positioning of the spike assembly at all times.

As shown in FIG. 4, an adjustable depth plate 122 is utilized to facilitate different length spike assemblies 101. Depth plate adjustment is achieved by manipulating the depth-plate adjustment screw 123 or other suitable means for securing the depth plate to a predetermined position.

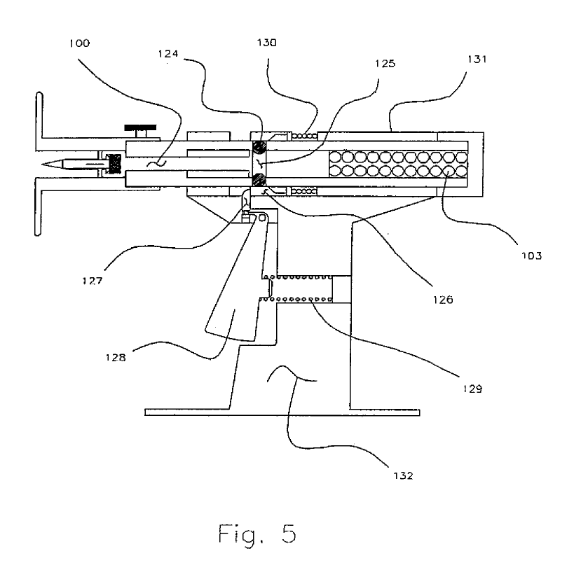

FIG. 5 shows the insertion gun in its loaded position ready for administering the spike assembly to the biological tissue.

FIG. 5 shows the piston 100 retracted to its loaded position and is held by one or more locking ball bearings 124. The bearings hold piston 100 in its preloaded position by means of seeding into the bearing slot 125 of the piston. The bearings are held in position while the bearings are engaged into the bearing slot 125 by means of sliding sleeve 126. The sliding sleeve is held in its loaded position by means of a falling block 127 which is interconnected to the hand trigger mechanism 128.

Handle spring 129 pushes the trigger handle 128 outward which in turn raises the falling block 127 into its loaded position which prevents the sliding sleeve from advancing forward.

### Actuation

As shown in FIG. 5, actuation of the trigger mechanism allows the grommet assembly 101 to be advanced at a velocity and force capable of penetrating biological skin and bone tissues with minimum recoiling.

Triggering function is achieved in the following manner. Physical pressure is applied to trigger handle 128 to overcome spring pressure 129. Movement of the trigger handle pulls falling block 127 down which in turn allows sliding sleeve 126 to advance forward by means of sleeve pressure spring 130. When sliding sleeve is in its forward position as shown in FIG. 4, clearance for the locking ball bearing 124 is made. Compression spring 103 applies spring force to piston 100 which in turn forces the locking ball bearings 124 to move from the piston bearing slot 125 to the clearance as provided by the sliding sleeve 126. During this transition the bearings traverse the bearing holes within the inserter body 131.

The inserter body 131 as in FIG. 5 is suitably mounted to a suitably designed handle 132 for facilitating ease of use.

### Grommets

Different configurations of devices may be suitably attached to the grommet proximal end 110 and attachment flange 118.

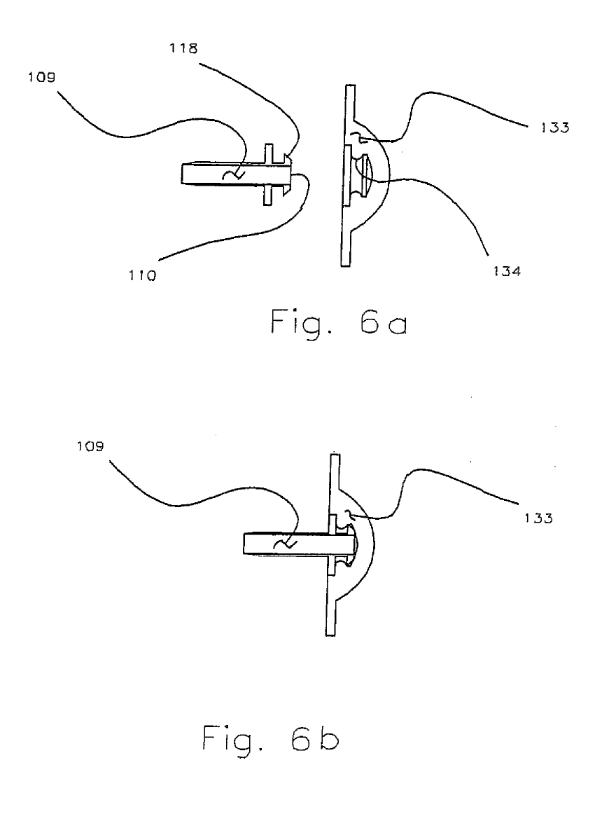

As shown in FIG. 6a, a plug cap 133 made of semi-rigid polymeric material is later secured to the grommet 109. The plug cap 133 consists of suitable geometry which allows ease of installation and removal while maintaining a fluid or gas cap seal 134. Additionally the gas cap seal serves as a retention device fixed to the grommet shown in FIG. 6b.

Other grommet seal configurations can be applied to the grommet proximal end 110.

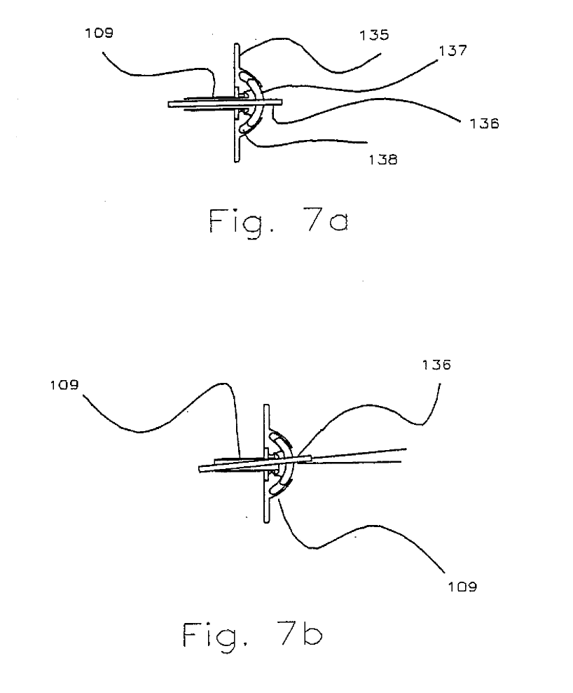

As shown in FIG. 7a, a polymeric cap 135 is shown in position on the grommet 109 and supports geometry to accept a movable catheter insert 136. The movable catheter insert 136 is comprised of a dome 137 which interfaces the polymeric cap receptacle 138. Further attributes of the polymeric cap receptacle 138 allow resistive interference to the movable catheter insert 136 to sustain the desired proper positioning and providing an air/gas seal in the surrounding area.

The catheter insert 136 can be positioned off the linear axis of the grommet to a degree proportional to the length of the grommet 109 as shown in FIG. 7b the maximum angle can be determined by the outer dimension of the catheter, the grommet inner dimension and the overall length of the grommet. Alternatively, the catheter insert fits snuggly in the grommet 109 and achieves a good air/gas seal.

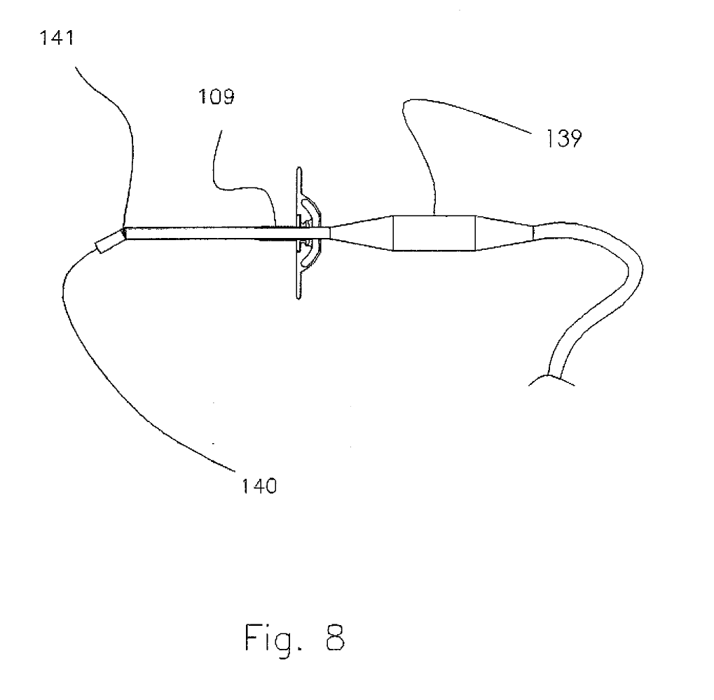

Other instruments having a movable and directionally controlled tip may be incorporated into the grommet geometry to facilitate the internal surgical procedure. As shown in FIG. 8, instrument 139 is inserted into the grommet 109 such that the instrument tip 140 is positioned off axis by means of the internal hinge mechanism of the instrument 141.

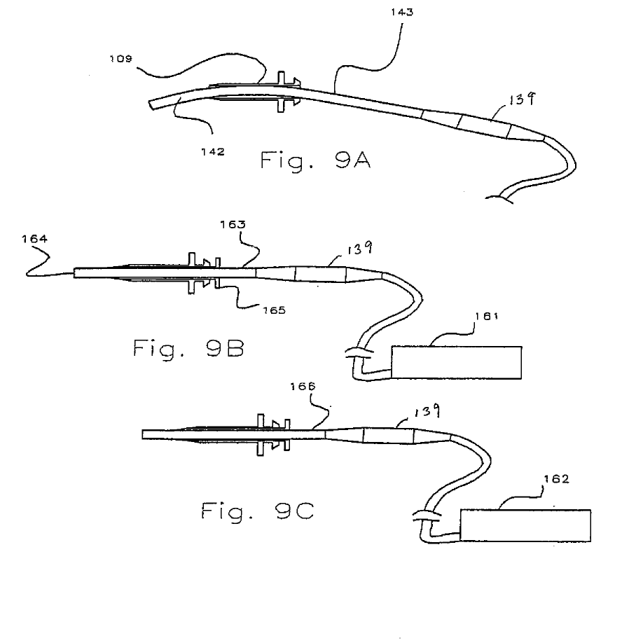

Another instrument can be used in conjunction with the grommet 109. In a preferred embodiment, a curved cannula 142 shown in FIG. 9a is part of the preferred curved tip instrument 143 positioned in its transitional state through the grommet 109.

FIG. 9b illustrates the instrument 139 attached to an ultrasound device 161. The ultrasound probe located at the tip of the inserter device will first guide the instrument into the lateral ventricle. Upon withdrawing the unit, a conduit sleeve 165 of Mylar or similar material that originally surrounded the ultrasound probe will remain in the lateral ventricle to act as a tunnel to guide instruments into the lateral ventricle. The sheath will have a metric ruler on its surface to help guide the proper depth of insertion of the various instruments including an irrigation/aspiration unit to control the temperature of the brain. The Mylar conduit sleeve may have one or more vent holes 170.

In the application of the ultrasound unit to other areas of the body, the ultrasound probe may be embedded with piezoelectric sound emitting chips to facilitate its intra-tissue localization via ultrasonic receivers placed outside body tissues or on the surface of the skin.

Ultrasound localization may be derived from 3-dimensional scanning combined with fiberoptic endoscopy, thereby giving more accurate localization of the device within the brain or other space within the body.

FIG. 9c illustrates a probe 166 attached to a fiberoptic camera system 162, or an optical scanning device, or a light and laser scanning device such as laser interferometry and other imaging modalities for the purpose of analyzing the anatomical and functional properties of the nearby tissues.

FIG. 9d illustrates an irrigating-aspiration system with temperature and pressure sensors 156 embedded in the tip of the unit 157. The irrigating/aspirating probe may be properly vented with one or more side vents to improve circulation of fluid to prevent total occlusion 174. One or more bypass vents may be located in the ventricle, the subarachnoid space, or external to the calvarium or other tissue organs.

FIG. 9e illustrates the device with use of deployable and interchangeable microsurgical tools and robotic endosurgical devices 171.

Further securing of a grommet seal can be accomplished by the incorporation of a vacuum seal made of rigid/semi-rigid polymeric or metallic material.

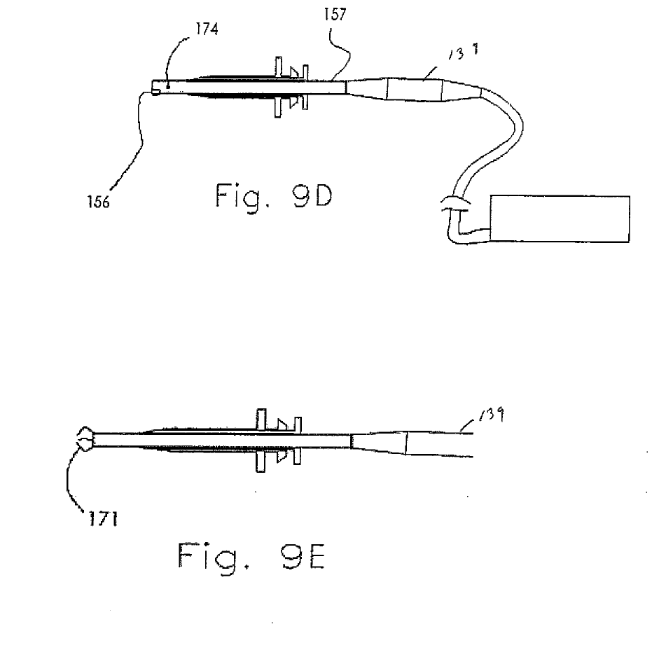

As shown in FIG. 10, a preferred means is shown comprising of a vacuum chamber 144 within the suction ring seal 145 which in turn is seated on grommet 109. A vacuum tube 146 provides a vacuum conduit from the vacuum chamber 144 to the vacuum source. A sterile drape 147 can be suitably designed to interact with the suction ring seal 145 by means of a suitable geometry such as a locking ring 148.

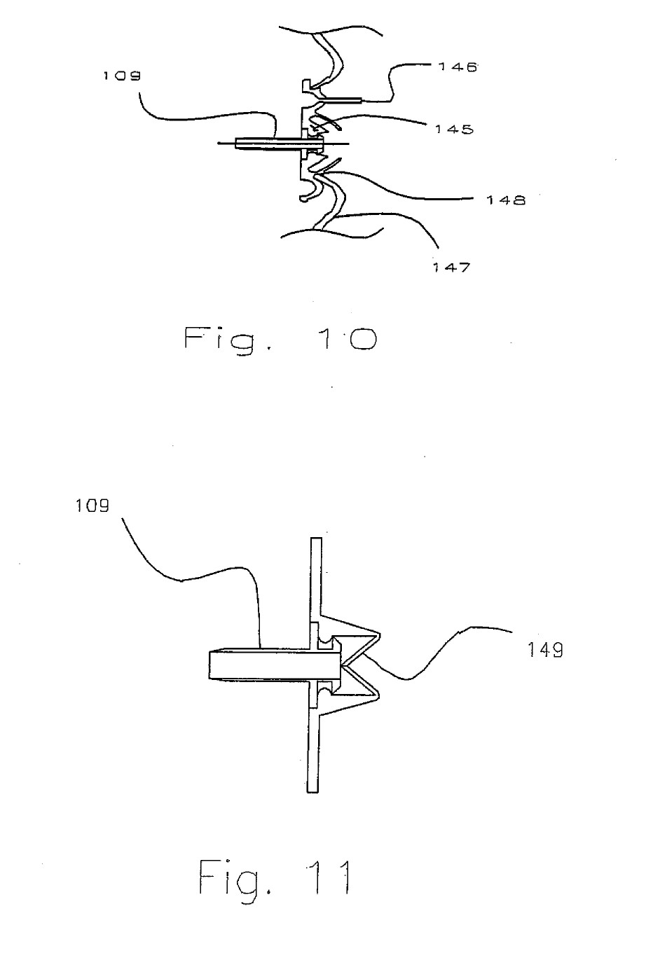

In conjunction with the grommet 109 supports, a reed-type seal 149 can be installed which allows case of entry and removal of instruments in a sterile manner without secondary intervention as in FIG. 11.

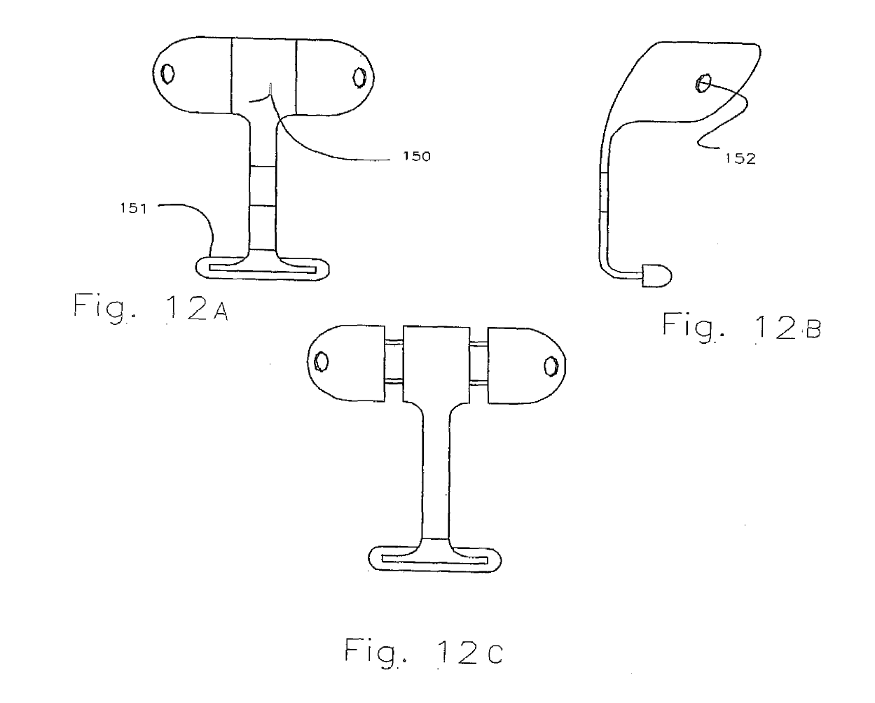

FIG. 12 depicts a location template 150 having the ability to adjust the guide hole 152 from the mouthpiece 151 that uses the teeth and/or gums of the maxilla for fixation and reference.

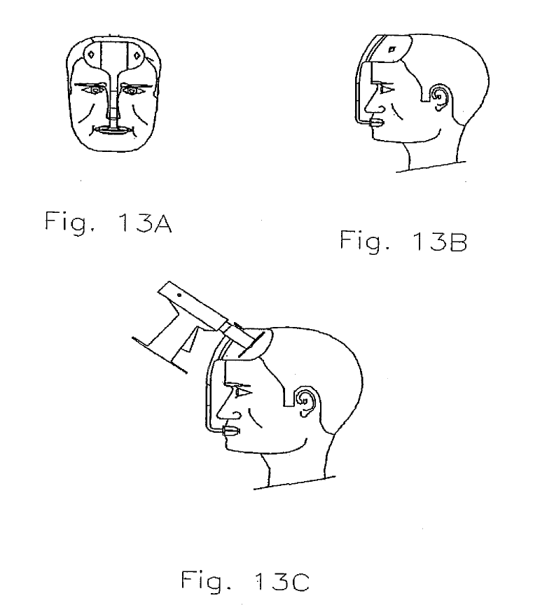

FIG. 13 shows the location of the template when applied to a patient for locating the position to which the grommet is to be positioned through the guide hole 152. The guide hole is of sufficient thickness to give stability to the inserter assembly. Additional fixation points such as the external auditory canal, the orbital rim, the mandible or temporo-mandibular joint can be added as fixation points connected to the location template 150. Once the grommet is in place in the skull, the template is removed from the surgical site.

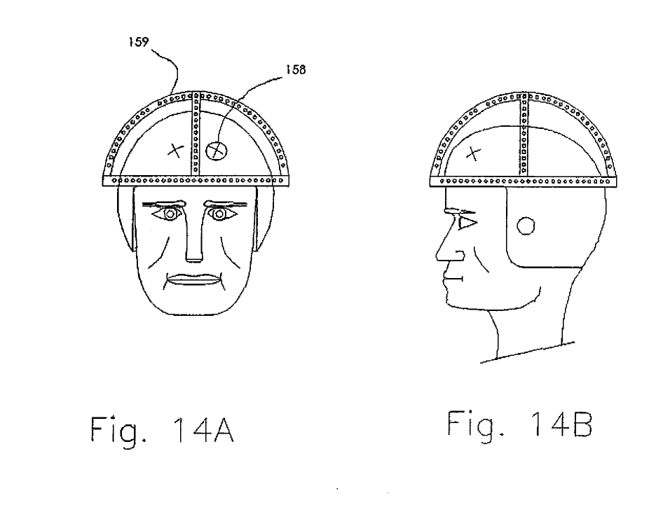

FIGS. 14A-B depict alternate means to locate a suitable position for the grommet location by use of a helmet. In this configuration, the helmet is strapped to the jaw, and landmarks include the external auditory canals, the mandible, the maxilla and the orbital rims. The helmet itself may immobilize the inserter unit once it enters the head. The helmet may facilitate the preservation of sterility and separate the surgical field from contaminants and scalp hair and other tissues. The fixation helmet FIGS. 14a and 14b may support and stabilize a stereotactic unit with X, Y, and Z co-ordinates for accurate localization of internal structures of the brain. Through openings in the helmet 158, the inserter can be aimed with the use of the stereotactic co-ordinates. Frontal and lateral views of the stereotactic device 159 are illustrated in FIGS. 14a and 14b.

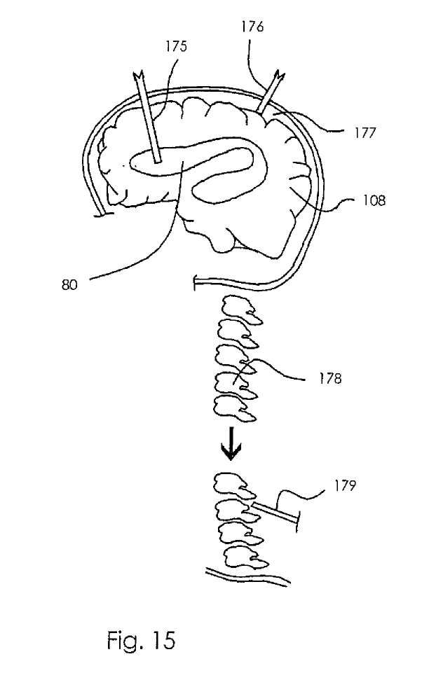

FIG. 15 illustrates the use of a vent elsewhere in the CNS, such as the subarachnoid space of the lumbar region 179, to allow for proper flow of fluid when doing irrigation/aspiration, placement of other treatment modalities including steroids, anti-inflammatories, antibiotics, antifungals, anti-cancer medications, and others therapies. Also illustrated are inserter probes 175 and 176 in the lateral ventricle 80 and the subarachnoid space 177 overlying the cortex of the brain 108. The vertebral column is represented by 178.

Although there has been hereinabove described a specific method and medical device for rapid and accurate entry through soft tissue and bone in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

1. A method for entering bone tissue of a body, said method comprising:
using a compression spring to inject a spike with a portal grommet thereabout through a calvaria; and
removing the spike and leaving the portal grommet inserted in the calvaria, the portal grommet providing intracranial access.

2. The method according to claim 1 further comprising using a stop mechanism to inject the spike through the calvaria to a predetermined depth.

3. The method according to claim 1 further comprising:
dispensing a helmet on a scalp; and
aiming an inserter using a stereotactic unit and thereafter operating the insertion with the compression spring to inject the spike and grommet.

4. The method according to claim 1 further comprising using a template to target a selected location on said scalp using teeth of a patient as a point of reference.

5. The method according to claim 1 further comprising irrigating and/or aspirating blood, cerebrospinal fluid (CSF) or other fluid or medications through the grommet.

6. The method according to claim 1 further comprising inserting a probe through said grommet.

7. The method according to claim 1 further comprising inserting an ultrasound device through the grommet and using the device to localize tissues of the brain, the subdural space, the subarachnoid space, and the lateral ventricle located within the central nervous system (CNS).

8. The method according to claim 1 further comprising inserting a fiberoptic camera through the grommet and using the fiberoptic camera to give direct visualization of the different compartments and tissues.

9. The method according to claim 1 further comprising inserting a piezoelectric sound emitting device through the grommet and using the piezoelectric device to facilitate intra-tissue and inter-tissue localization.

10. The method according to claim 1 further comprising determining a trajectory of the spike and grommet through the use of an ultrasound device for imaging tissue interfaces, the subdural space, the subarachnoid space, and the ventricular system for the purpose of visualization of the correct trajectory of the inserter.

11. The method according to claim 1 further comprising determining a trajectory of the spike and grommet through the use of a fiberoptic camera system to directly visualize the trajectory of the spike and grommet entering the subdural space, the subarachnoid space, the brain parenchyma, and the ventricular system in order to ensure accurate placement of the spike and grommet into a particular target, the camera system incorporating an optical scanning device and/or other light and laser scanning devices such as laser interferometry and other imaging modalities for the purpose of analyzing the anatomical and functional properties of the nearby tissues.

12. The method according to claim 1 further comprises using the template to determine a trajectory for insertion of an inserter into the calvaria, the template supporting and stabilizing a stereotactic unit.

13. A method for entering bone tissue of a body, said method comprising:
using a pneumatic device to inject a spike with a portal grommet thereabout through a calvaria; and
removing the spike and leaving the portal grommet inserted in the calvaria, the portal grommet providing intracranial access.

14. The method according to claim 13 further comprising using a stop mechanism to inject the spike through the calvaria to a predetermined depth.

15. The method according to claim 13 further comprising:
dispensing a helmet on the scalp; and
aiming the device using a stereotactic unit and thereafter operating the insertion with the compression spring to inject the spike and grommet.

16. The method according to claim 13 further comprising using a template to target a selected location on said scalp using teeth of a patient as a point of reference.

17. The method according to claim 13 further comprising irrigating and/or aspirating blood, cerebrospinal fluid (CSF) or other fluid or medications through the grommet.

18. The method according to claim 13 further comprising inserting a probe through said grommet.

19. The method according to claim 13 further comprising inserting an ultrasound device through the grommet and using the device to localize tissues of the brain, the subdural space, the subarachnoid space, and the lateral ventricle located within the central nervous system (CNS).

20. The method according to claim 13 further comprising inserting a fiberoptic camera through the grommet and using the fiberoptic camera to give direct visualization of the different compartments and tissues.

21. The method according to claim 13 further comprising inserting a piezoelectric sound emitting device through the grommet and using the piezoelectric device to facilitate intra-tissue and inter-tissue localization.

22. The method according to claim 13 further comprising determining a trajectory of the spike and grommet through the use of an ultrasound device for imaging tissue interfaces, the subdural space, the subarachnoid space, and the ventricular system for the purpose of visualization of the correct trajectory of the inserter.

23. The method according to claim 13 further comprising determining a trajectory of the spike and grommet through the use of a fiberoptic camera system to directly visualize the trajectory of the spike and grommet entering the subdural space, the subarachnoid space, the brain parenchyma, and the ventricular system in order to ensure accurate placement of the spike and grommet into a particular target, the camera system incorporating an optical scanning device and/or other light and laser scanning devices such as laser interferometry and other imaging modalities for the purpose of analyzing the anatomical and functional properties of the nearby tissues.

24. The method according to claim 13 further comprises using the template to determine a trajectory for insertion of an inserter into the calvaria, the template supporting and stabilizing a stereotactic unit.
